SEQUENCE LISTING (9 sequences)

SEQ ID NO: 1 - protein, 257 aa, Homo sapiens
MEHIQGAWKTISNGFGFKDAVFDGSSCISPTIVQQFGYQRRASDDGKLTDPSKTSNTIRVFLPNKQRTVVNVRNGMSLHDCLMKALKVRGLQPECCAVFRLLHEHKGKKARLDWNTDAASLIGEELQVDFLDHVPLTTHNFARKTFLKLAFCDICQKFLLNGFRCQTCGYKFHEHCSTKVPTMCVDWSNIRQLLLFNPSTIGDSGVPQLPSLTMRRMRESVSRMPVSSQHRYSTPHAFTFNTSSPSSEGSLSQRQRS

SEQ ID NO: 2 - protein, 149 aa, Homo sapiens
MEHIQGAWKTISNGFGFKDAVFDGSSCISPTIVQQFGYQRRASDDGKLTDPSKTSNTIRVFLPNKQRTVVNVRNGMSLHDCLMKALKVRGLQPECCAVFRLLHEHKGKKARLDWNTDAASLIGEELQVDFLDHVPLTTHNFARKTFLKL

SEQ ID NO: 3 - protein, 131 aa, Homo sapiens
MEHIQGAWKTISNGFGFKDAVFDGSSCISPTIVQQFGYQRRASDDGKLTDPSKTSNTIRVFLPNKQRTVVNVRNGMSLHDCLMKALKVRGLQPECCAVFRLLHEHKGKKARLDWNTDAASLIGEELQVDFL

SEQ ID NO: 4 - protein, 99 aa, Homo sapiens
PSKTSNTIRVFLPNKQRTVVNVRNGMSLHDCLMKALKVRGLQPECCAVFRLLHEHKGKKARLDWNTDAASLIGEELQVDFLDHVPLTTHNFARKTFLKL

SEQ ID NO: 5 - protein, 81 aa, Homo sapiens
PSKTSNTIRVFLPNKQRTVVNVRNGMSLHDCLMKALKVRGLQPECCAVFRLLHEHKGKKARLDWNTDAASLIGEELQVDFL

SEQ ID NO: 6 - protein, 79 aa, Homo sapiens
NVRNGMSLHDCLMKALKVRGLQPECCAVFRLLHEHKGKKARLDWNTDAASLIGEELQVDFLDHVPLTTHNFARKTFLKL

SEQ ID NO: 7 - protein, 32 aa, Homo sapiens
LDWNTDAASLIGEELQVDFLDHVPLTTHNFAR

SEQ ID NO: 8 - protein, 18 aa, Homo sapiens
VRGLQPECCAVFRLLHEH

SEQ ID NO: 9 - protein, 15 aa, Homo sapiens
LQPECCAVFRLLHEH

(19) United States
(12) Patent Application Publication
(10) Pub. No.: US 2003/0170751 A1
(43) Pub. Date: Sep. 11, 2003

(54) Immunohistochemical assay for ras activity

(76) Inventors: Lawrence S. Sherman, Cincinnati, OH (US); Nancy Ratner, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 09/569,197

(22) Filed: May 11, 2000

Related U.S. Application Data
(60) Provisional application No. 60/133,811, filed on May 11, 1999.

(51) Int. Cl. G01N 33/574; G01N 33/53

(52) U.S. Cl. 435/7.23; 435/7.5

(57) ABSTRACT

The present invention generally relates to methods for diagnosis and prognosis of cancers and more particularly to methods of detecting activated ras oncogene in cells and tissues. This method makes use of a fusion protein construct comprising the ras oncogene binding domain (“RBD”) of the raf protein which is linked to GST. This forms a “raf-GST” construct that binds to the ras oncogene only when ras is activated, i.e., in the ras-GTP state.

### FIELD OF THE INVENTION

The present invention generally relates to methods for diagnosis and prognosis of cancers and more particularly to methods of detecting activated ras oncogene in cells and tissues. This method makes use of a fusion protein construct comprising the ras oncogene binding domain (“RBD”) of the raf protein which is linked to GST. This forms a “raf-GST” construct that binds to the ras oncogene only when ras is activated, i.e., in the ras-GTP state.

### BACKGROUND OF THE INVENTION

The ras gene was discovered as an oncogene of the Harvey (rasH) and Kirsten (rasK) rat sarcoma viruses. In humans, characteristic mutations in the cellular ras gene (c-ras) have been associated with many different types of cancers. These mutant alleles, which render Ras constitutively active, have been shown to transform cells, such as the murine cell line NIH 3T3, in culture.

The ras gene product binds to guanine triphosphate (GTP) and guanine diphosphate (GDP) and hydrolyzes GTP to GDP. It is the GTP-bound state of Ras that is active. An accessory molecule, GTPase-activating protein (GAP) also binds to Ras and accelerates the hydrolysis of GTP. The ras proto-oncogene requires a functionally intact raf -1 proto-oncogene in order to transduce growth and differentiation signals initiated by receptor and non-receptor tyrosine kinases in higher eukaryotes. Activated Ras is necessary for the activation of the c-raf-1 proto-oncogene, but the biochemical steps through which Ras activates the Raf-1 protein (Ser/Thr) kinase are not well characterized. Wild type Ras binds specifically to the aminoterminal regulatory segment of Raf-1 in a GTP dependent fashion. The binding of Ras to Raf is strongly dependent on the nature of the guanyl nucleotide bound to Ras.

Presently, the method used to evaluate the activity of ras involved a time consuming ³²P-radiolabel method. The present method would be simpler, less expensive and would not require the use of radioisotopes. This method would have use as (a) a method of determining the prognosis of a patient and (b) a method of following the course of action of certain drugs which effect ras activity.

Presently, the raf-RBD-GST construct is produced by Onyx Pharmaceuticals of California. The only publication of the use of this construct is the use in pulling out the protein involved with the construct and an antibody using a lysate of a large number of cells.

U.S. Pat. No. 5,582,995, Avruch et al., issued Dec. 10, 1996, discloses methods of screening for compounds which inhibit the direct binding of Ras to Raf. These are methods of screening for compounds that inhibit the direct binding of Ras or Raf -binding fragments thereof to Raf or Ras binding fragments thereof.

The invention features a method of evaluating a compound, e.g., for the ability to bind to zinc-finger domains, for signal-transduction-inhibiting properties, for cell proliferation inhibiting properties, for the ability to alter the cell cycle, or for the ability to inhibit a biological activity, e.g., the ability to bind to another protein, e.g., an oncogene protein or an oncogene protein substrate, of a zinc-finger domain containing protein, an oncogene protein, a cellular oncogene protein, or a proto-oncogene protein.

In another aspect, the invention features a method of evaluating a compound, e.g., for signal-transduction-inhibiting properties, for cell proliferation inhibiting properties, for the ability to alter the cell cycle, or for the ability to inhibit the biological activity, e.g., the ability to bind to another protein, e.g., an oncogene protein or an oncogene protein substrate, of a zinc-finger domain containing protein, an oncogene protein, cellular oncogene protein or proto-oncogene protein.

### DETAILED DESCRIPTION OF THE INVENTION

This invention pertains to methods for diagnosis and prognosis of cancers and more particularly to methods of detecting activated ras oncogene in cells and tissues. This method makes use of a fusion protein construct comprising the ras oncogene binding domain (“RBD”) of the raf protein which is linked to GST. This forms a “raf-GST” construct that binds to the ras oncogene only when ras is activated, i.e., in the ras-GP state.

The raf-GST may be incorporated into cells and tissues. Commercially available anti-GST antibodies (“GST-Ab”) then may be used to bind the GST portion of the raf-GST. A second antibody, an anti-“GST-Ab” antibody is then used to label the GST-Ab with an appropriate marker (e.g., radioisotope, fluorescent tag, etc.). The raf-GST is then quantified by the amount of marker detected.

The ras gene product binds to guanine triphosphate (GTP) and guanine diphosphate (GDP) and hydrolyzes GTP to GDP. It is the GTP-bound state of Ras that is active. An accessory molecule, GTPase-activating protein (GAP) also binds to Ras and accelerates the hydrolysis of GTP. The ras proto-oncogene requires a functionally intact raf-1 proto-oncogene in order to transduce growth and differentiation signals initiated by receptor and non-receptor tyrosine kinases in higher eukaryotes. The ras-binding fragment of Raf is made up of Raf residues 1-257

[MEHIQGAWKTISNGFGFKDAVFDGSSCISPTIVQQFGYQRRASDDGKLTDPSKTSNTI RVFLPNKQRTVVNVRNGMSLHDCLMKALKVRGLQPECCAVFRLLHEHKGKKARLD WNTDAASLIGEELQVDFLDHVPLTTHNFARKTFLKLAFCDICQKFLLNGFRCQTCGY KFHEHCSTKVPTMCVDWSNIRQLLLFNPSTIGDSGVPQLPSLTMRRMRESVSRMPVS SQHRYSTPHAFTFNTSSPSSEGSLSQRQRS (SEQ ID NO:1)] [Raf(1-257)].

Activated Ras is necessary for the activation of the c-raf -1 proto-oncogene, but the biochemical steps through which Ras activates the Raf -1 protein (Ser/Thr) kinase are not well characterized. Wild type Ras binds specifically to the aminoterminal regulatory segment of Raf-1 in a GTP dependent fashion. The binding of Ras to Raf is strongly dependent on the nature of the guanyl nucleotide bound to Ras.

Constitutive activation of ras proto-oncogene is a key step in the progression of numerous cancers. Aberrant ras activation can be due to either mutations in the ras gene or alterations in ras mediators. The ability to detect elevated ras activity in neoplastic and pre-neoplastic lesions with significantly aid in the early diagnosis and management of cancer. Current ras activity assays, however, are not practical for most clinical diagnostic applications.

The methods of the present invention includes using Raf, a Ras-binding fragment of Raf, e.g., an amino-terminal, non-catalytic fragment of Raf, e.g., Raf residues 1-257

[MEHIQGAWKTISNGFGFKDAVFDGSSCISPTIVQQFGYQRRASDDGKLTDPSKTSNTI RVFLPNKQRTVVNVRNGMSLHDCLMKALKVRGLQPECCAVFRLLHEHKGKKARLD WNTDAASLIGEELQVDFLDHVPLTTHNFARKTFLKLAFCDICQKFLLNGFRCQTCGY KFHEHCSTKVPTMCVDWSNIRQLLLFNPSTIGDSGVPQLPSLTMRRMRESVSRMPVS SQHRYSTPHAFTFNTSSPSSEGSLSQRQRS (SEQ ID NO:1)] [Raf(1-257)] to identify the activity of the Ras oncogene in a biologival sample such as cells, tissues, organs, fluids, lysates

Preferably, the fragment is a peptide containing residues 1-149 of Raf

[MEHIQGAWKTISNGFGFKDAVFDGSSCISPTIVQQFGYQRRASDDGKLTDPSKTSNTI RVFLPNKQRTVVNVRNGMSLHDCLMKALKVRGLQPECCAVFRLLHEHKGKKARLD WNTDAA SLIGEELQVDFLDHVPLTTHNFARKTFLKL (SEQ ID NO:2)] [Raf(1-149)] or a peptide containing residues 1 -131 of Raf

[MEHIQGAWKTISNGFGFKDAVFDGSSCISPTIVQQFGYQRRASDDGKLTDPSKTSNTI RVFLPNKQRTVVNVRNGMSLHDCLMKALKVRGLQPECCAVFRLLHEHKGKKARLD WNTDAASLIGEELQVDFL (SEQ ID NO:3)] [Raf(1-131)].

More preferably, the fragment is a peptide containing residues 51 -149 of Raf

[PSKTSNTIRVFLPNKQRTVVNVRNGMSLHDCLMKALKVRGLQPECCAVFRLLHEHK GKK ARLDWNTDAASLIGEELQVDFLDHVPLTTHNFARKTFLKL (SEQ ID NO:4)][Raf(51-149)]; a peptide containing residues 51-131 of Raf

[PSKTSNTIRVFLPNKQRTVVNVRNGMSLHDCLMKALKVRGLQPECCAVFRLLHEHK GKKARLDWNTDAASLIGEELQVDFL (SEQ ID NO:5)] [Raf(51-131)]; a peptide containing residues 71-149 of Raf

[NVRNGMSLHDCLMKALKVRGLQPECCAVFRLLHEHKGKKARLDWNTDAASLIGEE LQVDFLDHVPLTTHNFARKTFLKL (SEQ ID NO:6)] [Raf(71-149)]; a peptide containing residues 112-143 of Raf[LDWNTDAASLIGEELQVDFLDHVPLTTHNFAR (SEQ ID NO:7)] [Raf(112-143)]; a peptide containing residues 88-105 of Raf [VRGLQPECCAVFRLLHEH (SEQ ID NO:8)] [Raf(88-105)]; or a peptide containing residues 91-105 [LQPECCAVFRLLHEH (SEQ ID NO:9)] [Raf(91-105)].

A “DNA construct” is a DNA molecule, or a clone of such a molecule, either single- or double-stranded that has been modified through human intervention to contain segments of DNA combined and juxtaposed in a manner that as a whole would not otherwise exist in nature.

The term “encoding” refers generally to the sequence information being present in a translatable form, usually operably linked to a promoter. A sequence is operably linked to a promoter when the functional promoter enhances transcription or expression of that sequence. The information is convertible using the standard, or a modified, genetic code. See, e.g., Watson et al, (1987) The Molecular Biology of the Gene (4th ed.) vols. 1&2, Benjamin, Menlo Park, Calif.

A “gene fusion” is a DNA construction (performed in vitro or in vivo) that results in the coding sequences from one gene (the “responder”) being transcribed and/or translated under the direction of the controlling sequences of another gene (the “controller”). Responder genes can be divided into two classes, reporters and effectors, with analytical or manipulative roles, respectively.

An “isolated” nucleic acid is a nucleic acid, e.g., an RNA, DNA, or a mixed polymer, which is substantially separated from other DNA sequences which naturally accompany a native human sequence, e.g., ribosomes, polymerases, and many other human genome sequences. The term embraces a nucleic acid sequence which has been removed from its naturally occurring environment, and includes recombinant or cloned DNA isolates and chemically synthesized analogs or analogs biologically synthesized by heterologous systems. A substantially pure molecule includes isolated forms of the molecule.

“Recombinant” polypeptides refer to polypeptides produced by recombinant DNA techniques; i.e., produced from cells transformed by an exogenous DNA construct encoding the desired polypeptide. “Synthetic” polypeptides are those prepared by chemical synthesis.

The term “recombinant” refers to a nucleic acid sequence that is not naturally occurring, or is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a single genetic entity comprising a desired combination of functions not found in the common natural forms. Restriction enzyme recognition sites are often the target of such artificial manipulations, but other site specific targets, e.g., promoters, DNA replication sites, regulation sequences, control sequences, or other useful features may be incorporated by design. A similar concept is intended for a recombinant, e.g., fusion, polypeptide.

A DNA “coding sequence” or a “nucleotide sequence encoding” a particular protein, is a DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, procaryotic sequences, cDNA from eucaryotic MRNA, genomic DNA sequences from eucaryotic (e.g., mammalian) DNA, and even synthetic DNA sequences. A transcription termination sequence will usually be located 3′ to the coding sequence.

A “promoter sequence” is a DNA regulatory region capable of binding RNA polymerase in a cell and initiating transcription of a downstream (3′ direction) coding sequence. For purposes of defining the present invention, the promoter sequence is bound at the 3′ terminus by the translation start codon (ATG) of a coding sequence and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eucaryotic promoters will often, but not always, contain “TATA” boxes and “CAT” boxes. Procaryotic promoters contain Shine-Dalgamo sequences in addition to the −10 and −35 consensus sequences.

DNA “control sequences” refer collectively to promoter sequences, ribosome binding sites, polyadenylation signals, transcription termination sequences, upstream regulatory domains, enhancers, and the like, which collectively provide for the transcription and translation of a coding sequence in a host cell.

A cell has been “transformed” by exogenous DNA when such exogenous DNA has been introduced inside the cell membrane. Exogenous DNA may or may not be integrated (covalently linked) to chromosomal DNA making up the genome of the cell. In procaryotes and yeasts, for example, the exogenous DNA may be maintained on an episomal element, such as a plasmid. With respect to eucaryotic cells, a stably transformed cell is one in which the exogenous DNA has become integrated into the chromosome so that it is inherited by daughter cells through chromosome replication. This stability is demonstrated by the ability of the eucaryotic cell to establish cell lines or clones comprised of a population of daughter cell containing the exogenous DNA.

The DNA compositions of this invention may be derived from genomic DNA or cDNA, prepared by synthesis or may be a hybrid of the various combinations. Recombinant nucleic acids comprising sequences otherwise not naturally occurring are also provided by this invention. An isolated DNA sequence includes any sequence that has been obtained by primer or hybridization reactions or subjected to treatment with restriction enzymes or the like.

Novel DNA sequences, such DNA sequences as parts of expression cassettes and vectors, as well as their presence in cells are provided, where the novel sequences comprise domains that do not naturally exist together.

The present methods rely on the observation that the minimal ras binding domain (RBD”) of raf1 (“Raf1-RBD”; amino acids 51 through 131; [Raf (51-131)]

[TDPSKTSNTIRVFLPNKQRTVVNVRNGMSLHDCLMKALKVRGLQPECCAVFRLLHE HKGKKARLDWNTDAASLIGEELQVD (SEQ ID NO:2)]), a serine/threonine kinase, can be used as a probe for ras activity. Raf1-RBD binds to Ras-GTP with high affinity and to Ras-GDP with low affinity. The present methods adapt the use of Raf1-RBD linked to an epitope tag glutathione-S-transferase (GST). Epitope tags other than GST could also be used. The raf1-RBD-GST is then used as immunocytochemical and immunohistochemical probe to precisely identify cells with elevated ras both in vitro and in tissue sections.

TABLE 1
Three- and One-Letter Abbreviations (common) for amino acids:

Asp = D = Aspartic Acid
Ala = A = Alanine
Arg = R = Arginine
Asn = N = Asparagine
Cys = C = Cysteine
Gly = G = Glycine
Glu = E = Glutamic Acid
Gln = Q = Glutamine
His = H = Histidine
Ile = I = Isoleucine
Leu = L = Leucine
Lys = K = Lysine
Met = M = Methionine
Phe = F = Phenylalanine
Pro = P = Proline
Ser = S = Serine
Thr = T = Threonine
Trp = W = Tryptophan
Tyr = Y = Tyrosine
Val = V = Valine

TABLE 2
Conservative Amino Acid Replacements

| For Amino Acid | Code | Replace With: |
|---|---|---|
| Alanine | A | D-Ala, Gly, Aib, B-Ala, Acp, L-Cys, D-Cys |
| Arginine | R | D-Arg, Lys, D-Lys, homo-Arg, D-homo-Arg, |
| | * | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, beta -Ala, Acp |
| Isoleucine | I | D-Ile, Val, D-Val, AdaA, AdaG, Leu, D-Leu, |
| | * | Met, D-Met |
| Leucine | L | D-Leu, Val, D-Val, AdaA, AdaG, Leu, D-Leu, |
| | * | Met, D-Met |
| Lysine | K | D-Lys, Arg, D-Arg, homo-Arg, D-homo-Arg, |
| | * | Met, D-Met, Ile, D-Ile, Orn, D-Orn |
| Methionine | M | D-Met, S-Me-Cys, Ile, D-Ile, Leu, D-Leu, |
| | * | Val, D-Val |
| Phenylalanine | F | D-Phe, Tyr, D-Thr, L-Dopa, His, D-His, |
| | * | Trp, D-Trp, Trans-3,4, or 5-phenylproline, |
| | * | AdaA, AdaG, cis-3,4, or 5-phenylproline, |
| | * | Bpa, D-Bpa |
| Proline | P | D-Pro, L-I-thioazolidine-4-carboxylic |
| | * | acid, D-or L-1-oxazolidine-4-carboxylic |
| | * | acid (Kauer, U.S. Pat. No. (4,511,390) |
| Serine | S | D-Ser, Thr, D-Thr, allo-Thr, Met, D-Met, |
| | * | Met(O), D-Met(O), L-Cys, D-Cys |
| Threonine | T | D-Thr, Ser, D-Ser, allo-Thr, Met, D-Met, |
| | * | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, |
| | * | AdaA, AdaG |

In its basic form, cells are fixed and permeablized, blocked with a buffer that prevents nonspecific binding of GST, then incubated with RBD-GST. After incubating with an anti-GST antibody and a biotinylated secondary antibody, RBD-GST is detected with avidin conjugated to a fluorochrome (e.g., BODIPYL-FL), or to horseradish peroxidase (HRP) followed by development with the HRP substrate, diaminobenzoate (DAB).

The methods of the present invention utilize a peptide containing at least 4 or more amino acids with at least 80% sequence identity to an amino acid sequence of Raf (1-257). In preferred embodiments, the amino acid sequence of Raf is the amino acid sequence of Raf(1-149), Raf(51-149), or Raf(51-131). A peptide 4 to 15 amino acids with at least 95% sequence identity to the amino acid sequence of Raf(51-131) is also within the invention. Preferably, the peptide shares 100% sequence identity with the amino acid sequence of Raf(51-131).

“Identity”, as used herein, refers to the subunit sequence similarity between two polymeric molecules, e.g., two peptide molecules. When a subunit position in both of the two molecules is occupied by the same monomeric subunit, e.g., if a position in each of two peptides is occupied by serine, then they are identical at that position. The identity between two sequences is a direct function of the number of matching or identical positions, e.g., if half (e.g., 5 positions in a polymer 10 subunits in length), of the positions in two peptide or compound sequences are identical, then the two sequences are 50% identical; if 90% of the positions, e.g., 9 of 10, are matched, the two sequences share 90% sequence identity. By way of example, the amino acid sequences VRGLQP and HAFLQP share 50% sequence identity.

Construction of Gst Fusion Proteins

Raf1-RBD-GST fusion proteins can be made by any method known in the art. A chimeric gene encoding a GST fusion protein can be constructed by fusing DNA encoding a peptide or peptide fragment to the DNA encoding the carboxyl terminus of GST (see e.g., Smith et al., 1988, Gene 67:31). The fusion construct, can be transformed into a suitable expression system. The fusion proteins can be purified by methods known to those skilled in the art, including purification by glutathione affinity chromatography. The purity of the product can be assayed by methods known to those skilled in the art, e.g., gel electrophoresis.

Fragments of Raf which bind Ras can be made by methods known to those skilled in the art. For example, a DNA fragment which expresses a putative Ras-binding Raf fragment can be fused to GST (as described herein), the fusion protein immobilized by binding to glutathione-SEPHAROSE, and the ability of the fusion protein to bind Ras or a fragment thereof determined.

The DNA sequences encoding Raf1 amino acid residues is preferably generated by the polymerase chain reaction (PCR).

The term “fragment”, as applied to a peptide, will ordinarily be at least about 10 amino acids, usually about 20 contiguous amino acids, preferably at least 40 contiguous amino acids, more preferably at least 50 contiguous amino acids, and most preferably at least about 60 to 80 or more contiguous amino acids in length. Such peptides can be generated by methods known to those skilled in the art, including proteolytic cleavage of the protein, de novo synthesis of the fragment, or genetic engineering.

Analogs can differ from the native peptides of Ras or Raf by amino acid sequence, or by modifications which do not affect the sequence, or by both. Preferred analogs include peptides whose sequences differ from the wild-type sequence (i.e., the sequence of the homologous portion of the naturally occurring peptide) only by conservative amino acid substitutions, preferably by only one, two, or three, substitutions, for example, substitution of one amino acid for another with similar characteristics (e.g., valine for glycine, arginine for lysine, etc.) or by one or more non-conservative amino acid substitutions, deletions, or insertions which do not abolish the peptide's biological activity. Table 2 lists a number of conservative amino acid substitutions.

Modifications (which do not normally alter primary sequence) include in vivo or in vitro chemical derivitization of peptides, e.g., acetylation or carboxylation. Also included are modifications of glycosylation, e.g., those made by modifying the glycosylation patterns of a peptide during its synthesis and processing or in further processing steps, e.g., by exposing the peptide to enzymes which affect glycosylation e.g., mammalian glycosylating or deglycosylating enzymes. Also included are sequences which have phosphorylated amino acid residues, e.g., phosphotyrosine, phosphoserine, or phosphothreonine.

The invention includes analogs in which one or more peptide bonds have been replaced with an alternative type of covalent bond (a “peptide mimetic”) which is not susceptible to cleavage by peptidases. Where proteolytic degradation of the peptides following injection into the subject is a problem, replacement of a particularly sensitive peptide bond with a noncleavable peptide mimetic will make the resulting peptide more stable and thus more useful as a reagent. Such mimetics, and methods of incorporating them into peptides, are well known in the art. Similarly, the replacement of an L-amino acid residue is a standard way of rendering the peptide less sensitive to proteolysis. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4,-dinitrophenyl. Blocking the charged amino- and carboxy-termini of the peptides would have the additional benefit of enhancing passage of the peptide through the hydrophobic cellular membrane and into the cell.

As used herein, “polynucleotide” refers to a polymer of deoxyribonucleotides or ribonucleotides, in the form of a separate fragment or as a component of a larger construct. DNA encoding the polypeptide of the invention can be assembled from cDNA fragments or from oligonucleotides that provide a synthetic gene that is capable of being expressed in a recombinant transcriptional unit. Polynucleotide sequences of the invention include DNA, RNA and cDNA sequences.

DNA sequences of the invention can be obtained by several methods. For example, the DNA can be isolated using hybridization procedures that are well known in the art. These include, but are not limited to: 1) hybridization of probes to genomic or cDNA libraries to detect shared nucleotide sequences; 2) antibody screening of expression libraries to detect shared structural features and 3) synthesis by the polymerase chain reaction (PCR).

Hybridization procedures are useful for the screening of recombinant clones by using labeled mixed synthetic oligonucleotide probes where each probe is potentially the complete complement of a specific DNA sequence in the hybridization sample that includes a heterogeneous mixture of denatured double-stranded DNA. For such screening, hybridization is preferably performed on either single-stranded DNA or denatured double-stranded DNA.

Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA clone by the hybridization of the target DNA to that single probe in the mixture which is its complete complement (Wallace, et al., Nucleic Acid Research, 9:879, 1981).

The development of specific DNA sequences encoding Ras can also be obtained by: 1) isolation of double-stranded DNA sequences from the genomic DNA; 2) chemical manufacture of a DNA sequence to provide the necessary codons for the polypeptide of interest; and 3) in vitro synthesis of a double-stranded DNA sequence by reverse transcription of mRNA isolated from a eukaryotic donor cell. In the latter case, a double-stranded DNA complement of mRNA is eventually formed which is generally referred to as cDNA. Of these three methods for developing specific DNA sequences for use in recombinant procedures, the isolation of genomic DNA isolates is the least common. This is especially true when it is desirable to obtain the microbial expression of mammalian polypeptides due to the presence of introns.

Peptides of the Raf1-RBD-GST can be used as antigens to immunize animals for the production of polyclonal antisera using standard protocols. To produce monoclonal antibodies, antibody-producing cells from the challenged animal can be immortalized (e.g. by fusion with an immortalizing fusion partner) to produce monoclonal antibodies. Monoclonal antibody-producing hybridomas can then be screened for antibody binding.

Antibodies directed against specific antigens may be detected by any of several methods known to those skilled in the art, e.g., by using an Ouchterlony double diffusion assay or an enzyme-linked immunoabsorbent assay (ELISA). In double diffusion assays, antigen and antibodies are placed in separate wells cut in a matrix, e.g., agarose on the surface of a glass plate. The contents of both wells diffuse through the matrix in all directions. Where the diffusing antigen and antigen-specific antibodies meet, a precipitin line forms. ELISA involves coating a substrate, e.g., well in a plastic dish, with a purified antigen. Serum to be tested is then added to the well. If present, antigen specific antibodies attach to the antigen coating the well. Non-binding material is washed away and a marker enzyme e.g., horse radish peroxidase or alkaline phosphatase, coupled to a second antibody directed against the antigen-specific primary antibody is added in excess and the nonadherent material is washed away. Finally the enzyme substrate is added to the well and the enzyme catalyzed conversion is monitored as indicative of presence of the antigen.

To produce monoclonal antibodies, antibody-producing cells from the challenged animal can be immortalized (e.g. by fusion with an immortalizing fusion partner) to produce monoclonal antibodies. Monoclonal antibody-producing hybridomas can then be screened for antibody binding as described above.

The invention can employ not only intact monoclonal or polyclonal antibodies, but also an immunologically-active antibody fragment, for example, a Fab or (Fab)₂ fragment; an antibody heavy chain, an antibody light chain; a genetically engineered single-chain Fv molecule (U.S. Pat. No. 4,946,778); or a chimeric antibody, for example, an antibody which contains the binding specificity of a murine antibody, but in which the remaining portions are of human origin.

The term “isolated” means any Ras polypeptide of the present invention, or any gene encoding a Ras polypeptide, which is essentially free of other polypeptides or genes; respectively, or of other contaminants with which the Ras polypeptide or gene might normally be found in nature.

As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibodies that bind to the Ras polypeptide of the invention can be prepared using an intact polypeptide or fragments containing small peptides of interest as the immunizing antigen. The polypeptide or a peptide such as Sequence ID No. 1 used to immunize an animal can be derived from translated cDNA or chemical synthesis which can be conjugated to a carrier protein, if desired. Such commonly used carriers that are chemically coupled to the peptide include keyhole limpet hemocyanin (KLH), thyroglobulin, bovine serum albumin (BSA), and tetanus toxoid. The coupled peptide is then used to immunize the animal (e.g., a mouse, a rat, or a rabbit).

If desired, polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See for example, Coligan, et al., Unit 9, Current Protocols in Immunology, Wiley Interscience, 1991, incorporated by reference).

It is also possible to use the anti-idiotype technology to produce monoclonal antibodies that mimic an epitope. For example, an anti-idiotypic monoclonal antibody made to a first monoclonal antibody will have a binding domain in the hypervariable region that is the “image” of the epitope bound by the first monoclonal antibody. Thus, in the present invention, an anti-idiotype antibody produced from an antibody that binds to the synthetic peptide of the invention can bind to the site on Ras which binds to Raf1-RBD, thereby preventing Ras from binding to and phosphorylating Raf1-RBD.

Polynucleotide sequences encoding the polypeptide or the synthetic peptide (SEQ ID NO:1) of the invention can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art. Such vectors are used to incorporate DNA sequences of the invention.

DNA sequences encoding the polypeptides can be expressed in vitro by DNA transfer into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, in other words when the foreign DNA is continuously maintained in the host, are known in the art.

In the present invention, the Ras polynucleotide sequences may be inserted into a recombinant expression vector. The term “recombinant expression vector” refers to a plasmid, virus or other vehicle known in the art that has been manipulated by insertion or incorporation of the genetic sequences. Such expression vectors contain a promoter sequence that facilitates the efficient transcription of the inserted genetic sequence of the host. The expression vector typically contains an origin of replication, a promoter, as well as specific genes that allow phenotypic selection of the transformed cells. The DNA segment can be present in the vector operably linked to regulatory elements, for example, a promoter.

The vector may include a phenotypically selectable marker to identify host cells that contain the expression vector. Examples of markers typically used in prokaryotic expression vectors include antibiotic resistance genes for ampicillin (beta-lactamases), tetracycline and chloramphenicol (chloramphenicol acetyltransferase). Examples of such markers typically used in mammalian expression vectors include the gene for adenosine deaminase (ADA), aminoglycoside phosphotransferase (neo, G418), dihydrofolate reductase (DHFR), hygromycin-B-phosphotransferase (HPH), thymidine kinase (TK), and xanthine guanine phosphoribosyltransferse (XGPRT,gpt).

Transformation of a host cell with recombinant DNA may be carried out by conventional techniques that are well known to those skilled in the art. Where the host is prokaryotic, such as E. coli, competent cells that are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be cotransformed with DNA sequences encoding the polypeptides of the invention, and a second foreign DNA molecule encoding a selectable phenotype, such as the herpes simplex thymidine kinase gene. Another method is to use a eukaryotic vital vector, such as simian virus 40 (SV40) or bovine papilloma virus, to transiently infect or transform eukaryotic cells and express the protein. (Eukaryotic Viral Vectors, Cold Spring Harbor Laboratory, Gluzman ed., 1982). Examples of mammalian host cells include COS, BHK, 293, and CHO cells.

Isolation and purification of host cell expressed polypeptide, or fragments thereof, provided by the invention, may be carried out by conventional means including preparative chromatography and immunological separations involving monoclonal or polyclonal antibodies.

The invention also provides a method for detecting a cell with Ras kinase activity or a cell proliferative disorder associated with Ras comprising contacting a cell component with Raf1-RBD N-terminal kinase activity with a reagent that binds to the component and measuring the interaction of the reagent with the component. Such reagents can be used to measure relative levels of Ras expression compared to normal tissue. The cell component can be nucleic acid, such as DNA or RNA, or protein. When the component is nucleic acid, the reagent is a nucleic acid probe or PCR primer. The interaction of a nucleic acid reagent with a nucleic acid encoding a polypeptide with Raf1-RBD N-terminal kinase activity is typically measured using radioactive labels, however, other types of labels will be known to those of skill in the art. When the cell component is protein, the reagent is typically an antibody probe. The probes are directly or indirectly detectably labeled, for example, with a radioisotope, a fluorescent compound, a bioluminescent compound, a chemiluminescent compound, a metal chelator or an enzyme. Those of ordinary skill in the art will know of other suitable labels for binding to the antibody, or will be able to ascertain such, using routine experimentation.

Preferably the probe for identification of a cell with Ras kinase activity is a Raf1-RBD protein. Ras activity within a cell is measured by the amount of phosphorylation of the Raf1-RBD protein probe. For example, the amount of Ras activity in a cell extract can be measured by mixing the extract with Raf1-RBD protein and adding a radioactive compound such as ³²p -ATP to the mixture of components. The amount of radioactivity that is incorporated into the Raf1-RBD probe is determined, for example by SDS-PAGE, and compared to a cell control containing Raf1-RBD and a normal level of Ras kinase activity.

The Raf1-RBD protein used in the method of detection of the Ras kinase described above may exist as a single protein unit or a fusion protein. The fusion protein preferably consists of Raf1-RBD and glutathione-S-transferase (GST) as a carrier protein. The Raf1-RBD nucleotide sequence is cloned 3′ to the carrier protein in an expression vector, such as pGEX or such derivatives as pGEX2T or pGEX3X, the gene is expressed, the cells are lysed, and the extract is poured over a column containing a resin or mixed directly with a resin to which the carrier protein binds. When GST is the carrier, a glutathione (GSH) resin is used. When maltose-binding protein (MBP) is the carrier, an amylose resin is used. Other carrier proteins and the appropriate binding resin will be known to those of skill in the art.

The materials of the invention are ideally suited for the preparation of a kit. The kit is useful for the detection of the level of a Raf1-RBD N-terminal kinase comprising an antibody which binds a Raf1-RBD N-terminal kinase or a nucleic acid probe which hybridizes to Ras nucleotide, the kit comprising a carrier means being compartmentalized to receive in close confinement therein one or more containers such as vials, tubes, and the like, each of the container means comprising one of the separate elements to be used in the assay. For example, one of the container means may comprise a monoclonal antibody of o the invention that is, or can be, detectably labeled. The kit may also have containers containing buffer(s) and/or a container comprising a reporter-means (for example, a biotin-binding protein, such as avidin or streptavidin) bound to a reporter molecule (for example, an enzymatic or fluorescent label).

The following examples are intended to illustrate but not limit the invention. While they are typical of those that might be used, other procedures known to those skilled in the art may alternatively be used.

### SEQUENCE LISTING

(1) GENERAL INFORMATION:

What is claimed is:

1. A method for identifying activated Ras oncogene, comprising:
a. incubating a biological sample with a buffer that prevents nonspecific binding of an epitope tag
b. incubating a biological sample with Ras-binding peptide having an amino acid sequence with 80-100% sequence identity to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and
c. detecting the conjugated Ras-binding peptide
wherein the incubations are carried out under conditions sufficient to allow the components to interact.

2. The method of claim 1, wherein the Ras-binding peptide has an amino acid sequence with 95% sequence identity to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9.

3. The method of claim 2, wherein the Ras-binding peptide is linked to an epitope tag.

4. The method of claim 3, wherein the epitope tag is glutathione-S-transferase.

5. The method of claim 4, wherein the Ras-binding peptide is detected with an anti-GST antibody and a biotinylated secondary antibody.

6. The method of claim 4, wherein the Ras-binding peptide is detected with avidin conjugated to a fluorochrome.
